(19) United States
(12) Patent Application Publication
(10) Pub. No.: US 2025/0010024 A1
(43) Pub. Date: Jan. 9, 2025

(54) CATHETER ASSEMBLY

(71) Applicant: ConvaTec Limited, Flintshire (GB)

(72) Inventors: Oliver Pfleger, Spital (GB); Jan Uhlar, Krcava (SK)

(21) Appl. No.: 18/891,501

(22) Filed: Sep. 20, 2024

Related U.S. Application Data
(63) Continuation of application No. PCT/GB2023/050675, filed on Mar. 20, 2023.

(30) Foreign Application Priority Data
Mar. 21, 2022 (GB) GB2203883.0

(51) Int. Cl. A61M 25/00

(52) U.S. Cl. CPC A61M 25/0017; A61M 25/002; A61M 2209/06

(57) ABSTRACT

A packaged catheter assembly that has a catheter having a proximal end for insertion into the body and a distal end, a pouch having a gripping element configured to engage the catheter, and a fluid reservoir configured to release wetting fluid into the pouch so as to wet the catheter. The catheter and fluid reservoir are arranged within the pouch. The pouch is configured to provide an opening through which the catheter may be withdrawn. The pouch has a wetting region in which wetting fluid preferentially collects distal to the opening. The gripping element is configured to direct the catheter through the wetting region during withdrawal of the catheter from the pouch. The catheter may be curved and may be a male urinary catheter. The catheter may be longer than the pouch.

### CROSS-REFERENCE TO RELATED DISCLOSURES

The present disclosure is a Continuation of International Application No. PCT/GB2023/050675 filed Mar. 20, 2023, and claims the benefit of GB2203883.0 filed Mar. 21, 2022, the disclosures of which being incorporated herein by reference in entirety.

### TECHNICAL FIELD OF THE INVENTION

The present invention relates to catheter assemblies. In particular the invention concerns packaged urinary catheter assemblies, and most particularly, but not exclusively packaged intermittent male urinary catheter assemblies, especially “closed” catheter assemblies.

### BACKGROUND TO THE INVENTION

Many people suffer from urinary problems that can make it difficult to pass urine. Intermittent catheter assemblies can provide a convenient and portable solution to this problem as a user is able to self-catheterise to relieve themselves as required. This reduces the effect of their urinary condition on their life and allows them to enjoy a relatively normal lifestyle.

When using a catheter, it is important that the catheter remains clean but when packaged this can be challenging as the catheter must be removed from the packaging. This can lead to the user touching a part of the catheter that is intended to be introduced into the body, such as the tube by accident while they attempt to remove the catheter from the packaging which can lead to a risk of infection during use. It is therefore important to provide packaging which is convenient and easy for the user to open and which presents the catheter to the user such that it can be removed from the packaging for use without accidentally touching a part of the catheter that should remain clean directly.

Many existing catheters attempt to maintain the sterility and cleanliness of the catheter prior to use by introducing a sleeve around the catheter. However, the sleeve itself can hinder the activation of the catheter surface prior to use, for example by blocking wetting fluid from coming into contact with the catheter. This can mean that the catheter is not activated prior to use which can lead to discomfort and injury during use.

It is also possible that the packaging containing the catheter and catheter are arranged such that if the user does not take care during removal of the catheter from the packaging, the entire catheter surface may not be activated by wetting fluid. This can also lead to discomfort and injury during use.

To provide discrete and portable catheter assemblies, packaging may be provided to contain the catheter and any other equipment such as wetting reservoirs or sachets and fluid collection bags before and/or after use. However, these items may become wet which makes handling and re-packaging of them difficult. In addition, where liquid is contained within a sachet, reservoir or bag within the packaging, any release (intentional or otherwise) of this may cause liquid to accidentally leak out of the packaging causing problems for the user.

In packaged catheter assemblies, fluid collection bags must be designed to collect a sufficient amount of liquid such that during use the bag does not overflow. This must also be balanced against the need of providing compact and discrete catheter assemblies that are suitable for intermittent use. Users can have issues when fluid collection bags are packaged inefficiently or interfere with other parts of the catheter assembly, such as the catheter or wetting fluid/wetting fluid reservoir during use.

It is an object of embodiments of the present invention to at least partially overcome or alleviate the above problems and/or to provide an improved catheter assembly.

### SUMMARY OF THE INVENTION

In broad terms, the present invention concerns a packaged catheter assembly comprising a pouch and a catheter, wherein the pouch is configured to contain the catheter within it.

According to a first aspect of the present invention there is provided a packaged catheter assembly comprising a pouch and a catheter, wherein the pouch is configured to contain the catheter within it and wherein the pouch comprises an absorbent material.

Advantageously the provision of absorbent material allows any fluid in the pouch to be absorbed. This reduces leakage from the pouch making it more convenient and comfortable for a user to use and carry. A leak could occur prior to use (e.g. if someone inadvertently bursts a fluid reservoir); during use (e.g. when shaking the pouch to distribute wetting fluid within it, or using the pouch as a surface on which to set up, or pack away the catheter assembly); or after use. In each of these situations, the provision of an absorbent material will be advantageous. Furthermore, as fluid in the pouch is absorbed by the absorbent material, then there is less fluid pooling within the bag and less fluid moving around within the pouch. This reduces noise associated with fluid sloshing within the bag and also makes the bag easier to carry as the weight distribution within it is more stable.

The absorbent material may be any suitable shape or size. The absorbent material may be any one of: an absorbent sachet; an absorbent layer; an absorbent strip. In one preferred embodiment the absorbent material is an absorbent layer. The absorbent layer may line at least part of an inside surface of the pouch. The absorbent layer may line at least 50%, 60%, 70%, 80%, or 90% of the inside surface of the pouch. The absorbent layer may line the whole inside surface of the pouch. Where the pouch comprises a front and/or rear wall, the absorbent layer may line at least part of the front and/or rear walls. The absorbent layer may line both the front and rear walls. The absorbent layer may comprise a single sheet of absorbent material folded to line both the front and rear walls of the pouch. Advantageously, the absorbent layer can be easily formed and added to the pouch construction as it can line the inside of the pouch and be folded inside.

The packaged catheter assembly may comprise a fluid collection bag configured to receive fluid from the catheter in use and as described in more detail below. Consequently, the packaged catheter assembly may be a closed catheter assembly. The absorbent material may be configured to absorb at least 10%, 20%, 30%, 40%, 50%, 60%, 70%, 80%, or 90% of the liquid capacity of the fluid collection bag. In one embodiment, the absorbent material is configured to absorb 100% of the liquid capacity of the fluid collection bag. Thus, the absorbent material ensures that it can absorb (all) liquid within the fluid collection bag if required to reduce sloshing of liquid within the pouch/fluid collection bag.

The packaged catheter assembly may comprise a fluid reservoir. The fluid reservoir may be configured to retain wetting fluid within it and as described in more detail below. The absorbent material may be configured to absorb at least 10%, 20%, 30%, 40%, 50%, 60%, 70%, 80%, or 90% of the liquid capacity of the fluid reservoir. In one embodiment, the absorbent material is configured to absorb 100% of the liquid capacity of the fluid reservoir. Thus, the absorbent material ensures that it can absorb liquid released from the fluid reservoir if required to dry components of the catheter assembly that must be handled by the user and to avoid liquid from leaking out of the pouch.

In another preferred embodiment the absorbent material may comprise an absorbent strip. The absorbent strip may be elongate. The absorbent strip may have a length and a width wherein the length is at least 2, 3, 4, 5, or 6 times larger than the width. The absorbent strip may be located adjacent to a mouth of the pouch. The absorbent strip may span the majority of the mouth of the pouch. The absorbent strip may span at least 50%, 60%, 70%, 80% or 90% of the width of the mouth. The absorbent strip may span no more than 90%, 80%, 70%, or 60% of the width of the pouch. The mouth may correspond to a part of the pouch which forms an opening to allow access to the contents of the pouch. The absorbent strip may line the mouth of the pouch. Two or more absorbent strips may be provided. For example, two opposing absorbent strips may be provided. One absorbent strip may be provided on each of two sides of the mouth of the pouch. Thus, absorbent strips may be used to help prevent fluid from leaking out of the pouch through the mouth of the pouch.

The absorbent material may comprise a sponge material, an absorbent polymer, or a super absorbent for example polyacrylic acid.

The pouch may comprise a flexible plastics material. The pouch may be formed from flexible plastic films or sheets. For example, polypropylene (PP), polyethylene terephthalate (PET), low density polyethylene (LDPE), metalized polyester (MET PET) or orientated polypropylene (OPP).

The pouch may comprise two walls (a front wall and a rear wall) and a peripheral seal between the two walls. The pouch may therefore define a generally open internal space bounded by the peripheral seal. The peripheral seal may provide a sterile seal. Thus, the pouch may maintain the sterility of its contents.

The peripheral seal may form one or more edges of the pouch. The peripheral seal may comprise a left lateral edge, right lateral edge, an upper edge and a base of the pouch. The left and right lateral edges may be defined as the left and right edges when viewing the pouch with the front wall in front of the rear wall, the base at the bottom and the upper edge at the top. The pouch may be any suitable shape such as circular, elliptical, triangular, tubular, prismatic or polygon, preferably the pouch is rectangular. The pouch may have a width defined as the distance between the left lateral edge and right lateral edge of the pouch of at least 60, 70, 80, 90 or 100 mm. The pouch may have a width of no more than 110, 100, 90, 80, or 70 mm. Preferably, the width is between 90 and 110 mm, and most preferably about 100 mm. The pouch may have a height defined as the distance between the upper edge and base of at least 100, 125, 150, 175, 200 or 225 mm. The pouch may have a height of no more than 250, 225, 200, 175, 150, or 125 mm. Preferably, the height is between 120 and 140 mm, and most preferably it is about 130 mm. The peripheral seal may comprise any one or more of: a weld; mechanical seal; heat seal; pressure seal; adhesive; solvent bond; ultraviolet bond; ultrasonic weld; laser weld; impulse weld; or friction weld. The peripheral seal may be re-sealable, for example through a resealable adhesive, an additional adhesive that is activated prior or during resealing, or a hook and loop/hook and hook fastener. Thus, the peripheral seal can be easily constructed to suit the specific needs of the pouch.

One edge of the pouch may comprise a folded edge. A folded edge may comprise a fold between the two walls. All edges other than the folded edge may be formed by the peripheral seal. The pouch may be formed by folding the two walls to form the folded edge. Thus, the pouch can be flexibly constructed from folding material if required.

The packaged catheter assembly may comprise a fluid reservoir. The fluid reservoir may be configured to retain wetting fluid within it. The fluid reservoir may be configured to release fluid into the pouch. The pouch may comprise a wetting region in which fluid released by the fluid reservoir preferentially collects. The wetting region may comprise a lower section of the pouch. The wetting region may comprise the bottom 50%, 40%, 30%, 20%, or 10% of the pouch, preferably 20%. Preferably the wetting region is adjacent to the base of the pouch. The pouch may be configured to form a pool of released fluid in the wetting region. Thus, the catheter may be more efficiently wetted by being passed through the pool of wetting fluid formed in the pouch.

The fluid reservoir may be arranged within the (sealed) pouch. The fluid reservoir may be provided as a separate device, or incorporated into the pouch material. The fluid reservoir may comprise a deformable, frangible or burstable sachet. Thus, the fluid reservoir is conveniently located within the pouch where it may directly release fluid into pouch to wet the catheter. A deformable, frangible or burstable reservoir enhances the convenience for a user as fluid can be easily released without additional tools and before the pouch is unsealed, thus reducing the chances of accidental wetting of the user. A sachet in particular can be easily handled on an assembly line and introduced more simply than attempting to handle fluid, so as to it into the package during manufacture.

The wetting fluid may activate the surface of the catheter and render it lubricious. The wetting fluid may be water, such as where the catheter has a hydrophilic surface. Any suitable wetting fluid may be used, for example polar (e.g. water-based) or non-polar (e.g. oil-based) wetting fluids depending on the surface properties of the catheter. The fluid reservoir may be configured to retain at least 5, 10, 15, 25, or 50 ml of wetting fluid.

The fluid reservoir may be a sachet. The fluid reservoir may be any suitable shape or size, such as square, rectangular, circular, ellipsoidal or irregularly shaped. Preferably, the fluid reservoir is elongate and rectangular. The fluid reservoir may have a length of at least 60%, 70% or 80% the height of the pouch. The fluid reservoir may have a length no more than 90%, 80% or 70% the height of the pouch. Preferably the fluid reservoir has a length of 80% the height of the pouch. The fluid reservoir may have a width of at least 5%, 10%, 15% or 20% the width of the pouch. The fluid reservoir may have a width of no more than 25%, 20%, 15% or 10% the width of the pouch. Preferably, the fluid reservoir has a width of 15% the width of the pouch. Thus, the fluid reservoir may be flexibly shaped and sized to conveniently fit within the pouch.

The fluid reservoir may be arranged within the pouch. The fluid reservoir may be arranged with its length parallel to one edge of the pouch. Preferably, the length of the fluid reservoir is parallel to the left and/or right lateral edge of the pouch. The fluid reservoir may be arranged adjacent to one edge of the pouch. Preferably, the fluid reservoir is arranged adjacent to the same edge of the pouch that the fluid reservoir's length is parallel to. Thus, the fluid reservoir is conveniently located against an edge of the pouch where it does not take up too much space.

The pouch may be formed from an opaque material. The pouch may have a textured or printed fabric-like appearance and/or feel. Where the pouch is opaque, the pouch may comprise a wetting marker to assist the user in locating the fluid reservoir within the pouch. Thus, the pouch is more comfortable and discreet for the user to carry in day-to-day life and the user can easily find and activate the fluid reservoir.

The pouch may comprise an interaction region defining a first point of contact for a user of the packaged catheter assembly. The interaction region may facilitate user access to the contents of the pouch. Actuation of the interaction region may create an opening in or near an upper edge of the peripheral seal. The catheter may be withdrawn through the opening. The catheter may be withdrawn through the opening. Thus, access to the pouch is provided at or near an upper edge of the pouch and as such, the fluid in the pool adjacent the base of the pouch is retained in the pouch while the catheter is withdrawn reducing the risk of spilling fluid outside the pouch.

The interaction region may comprise a breakable region of the peripheral seal. The method may comprise forming a breakable region in the peripheral seal. The breakable region may comprise a tear-away region which may comprise a tear line defining a line along which the pouch may be torn to at least partially separate the tear-away region from the remainder of the pouch, thereby creating an opening in the peripheral seal. The tear-away region may comprise a tear start. The tear start may be provided at a first end of the tear-away region. The tear start may comprise a notch, or area of weakness, in one or both of the walls of the pouch. The tear start may define the first part of the pouch to be torn. The tear-away region may comprise a tear stop. The tear stop may be provided at a second end of the tear-away region. The tear stop may act to prevent or reduce the likelihood of the tear-away region being completely separated from the remainder of the packaging. A tear line may be defined between the tear start and the tear stop. The tear line may be configured to preferentially tear. The tear line may comprise a weakened region of the pouch. The tear line may be formed by any suitable means, such as laser scoring. Thus, the breakable region provides a convenient and easy means to open the pouch with minimal effort and no additional equipment/tools.

The tear-away region may be substantially triangular in shape having a sloped upper edge. The tear-away region may be tapered. The tear-away region may extend upwards more on one side than the other, preferably the tear-away region extends upwards more on the side corresponding to the tear start. The tear-away region may extend upwards no more than 200%, 100%, 75%, or 50% more on one side than the other. The tear-away region may extend upwards at least 25%, 50%, 75%, 100%, or 150% more on one side than the other. Preferably, the tear-away region extends upwards 50% more on one side than the other. Thus, the tear-away region is conveniently shaped to guide the user to grasp it on one side to open the pouch and also provides sufficient material for the user to securely grasp it on that side.

The absorbent strip may be positioned adjacent the tear line. The absorbent strip may be positioned below the tear line. The absorbent strip may span a majority of the tear line, for example at least 50%, 60%, 70%, 80% or 90% of the length of the tear line. Thus, the opening formed along the tear line is next to the absorbent strip and the absorbent strip may then absorb fluid within the pouch before it leaves through the opening.

The interaction region may comprise an aperture. The aperture may be sized to allow a finger to pass through and grip the interaction region. Preferably, the aperture is located in the tear-away region. The aperture may be located on the same side of the pouch as the tear start. The aperture may be located adjacent to the tear start. The aperture may be any suitable shape, for example, circular, elliptical, square or irregularly shaped. Preferably, the aperture is circular. Thus, the user can more easily grasp the tear-away region and open the pouch.

The catheter may be a male catheter (i.e. a catheter for catheterisation of men). The catheter may be a female catheter (i.e. a catheter for catheterisation of women). The catheter may comprise a proximal end for insertion into the body and a distal end. The catheter assembly may comprise a sleeve. The sleeve may comprise a proximal end and a distal end. The distal end of the sleeve may be secured or attached either directly or indirectly to the distal end of the catheter. The sleeve may be configured to enclose a majority of the length of the catheter. The sleeve may comprise a flexible plastics material. The sleeve may be liquid impermeable. Alternatively, the sleeve may be liquid permeable. Where the sleeve is liquid permeable, it may be provided in a configuration in which it covers a majority of the length of the catheter. The sleeve may comprise a handling element. The handling element may be disposed at the proximal end of the sleeve. The handling element may be configured to control movement of the sleeve with respect to the catheter. The handling element may be annular. The handling element may be more rigid than the sleeve. The sleeve may comprise a thermoplastic polyurethane (TPU) or low-density polyethylene (LDPE). Thus, the sleeve is cheap and easy to produce and can be easily manipulated by the user during use.

Accordingly in one embodiment there is provided a packaged catheter assembly comprising a male urinary catheter having a proximal end for insertion into the body and a distal end, a pouch comprising a gripping element configured to engage the catheter, and a fluid reservoir configured to release wetting fluid into the pouch so as to wet the catheter, wherein: the catheter and fluid reservoir are arranged within the pouch; the pouch is configured to provide an opening through which the catheter may be withdrawn; the pouch comprises a wetting region in which wetting fluid preferentially collects; the wetting region is distal the opening; and the gripping element is configured to direct the catheter through the wetting region during withdrawal of the catheter from the pouch.

The pouch may comprise a gripping element. The gripping element may be configured to engage the sleeve. The gripping element may be configured to unfurl the sleeve along the length of the catheter. The gripping element may be configured to detach from the sleeve.

Thus, according to a broad aspect of the present invention, there is provided a packaged catheter assembly comprising a catheter having a proximal end for insertion into the body and a distal end, a sleeve enclosing the catheter, a pouch comprising a gripping element configured to engage the sleeve, and a fluid reservoir configured to release wetting fluid into the pouch so as to wet the catheter, wherein: the catheter and fluid reservoir are arranged within the pouch; and the gripping element is configured to unfurl the sleeve along the length of the catheter, and detach from the sleeve.

The gripping element may be configured to remain in the pouch.

Thus, according to a second aspect of the present invention, there is provided a packaged catheter assembly comprising a catheter having a proximal end for insertion into the body and a distal end, a sleeve enclosing the catheter, a pouch comprising a gripping element configured to engage the sleeve, and a fluid reservoir configured to release wetting fluid into the pouch so as to wet the catheter, wherein: the catheter and fluid reservoir are arranged within the pouch; and the gripping element is configured to unfurl the sleeve along the length of the catheter, detach from the sleeve, and remain in the pouch.

Consequently, the sleeve may be provided in a furled configuration. The gripping element may retain the sleeve in the furled configuration when within the pouch. Thus, the catheter is exposed within the pouch and may be directly wetted by the wetting fluid released into the pouch. Once wetted, the sleeve may be unfurled along the length of the catheter to protect the now wetted catheter from dirt and allow the user to handle the catheter via the sleeve and without coming into contact with the wetting fluid or catheter itself. In addition, as the gripping element detaches from the sleeve and remains in the pouch, it does not become an inconvenient loose object that the user must try to keep track of and dispose of.

In the furled configuration, the sleeve may cover a minority of the length of the catheter. In the furled configuration, the sleeve may cover parts of the catheter which are not required to pass into the body during use. In the furled configuration, the sleeve may cover no more than 40%, 30%, 20%, 10% or 5% of the length of the catheter. In the furled configuration, the sleeve may cover at least 5%, 10%, 20%, or 30% of the length of the catheter. Thus, the furled sleeve leaves the majority of the catheter uncovered and ready to be wetted within the pouch.

The gripping element may be positioned in an upper half of the pouch. The gripping element may be positioned at least 50%, 60%, 70% or 80% of the way up the height of the pouch, preferably about 60% to 70% of the way up the height of the pouch. The sleeve may be positioned above the gripping element in the pouch. This means that wetting fluid released into the pouch is not likely to wet the sleeve.

Once unfurled, the sleeve may be in an unfurled configuration. In the unfurled configuration, the sleeve may cover a majority of the length of the catheter. In the unfurled configuration, the sleeve may cover at least 50%, 60%, 70%, 80%, 90% or 95% of the length of the catheter. Thus, the unfurled sleeve provides good protection to the catheter from contact with the user or other objects which may render it dirty and unusable.

The gripping element may be configured to unfurl the sleeve as the catheter is withdrawn from the pouch. The gripping element may unfurl the sleeve by preventing movement of a part of the sleeve, and preferably one end of the sleeve. The gripping element may engage the sleeve adjacent to, or at, an end of the sleeve corresponding to an end of the catheter which is the last part of the catheter to be withdrawn from the pouch. Where the catheter is withdrawn from the pouch by its distal end, the gripping element may engage the proximal end of the sleeve. Where the catheter is withdrawn from the pouch by its proximal end, the gripping element may engage the distal end of the sleeve. Thus, the gripping element can ensure the sleeve is completely unfurled and conveniently unfurls the sleeve as the catheter is withdrawn from the pouch.

The gripping element may be positioned adjacent or along an edge of the pouch. The gripping element may extend into the pouch. The gripping element may be attached to the peripheral seal of the pouch. The gripping element may be positioned on a lateral edge of the peripheral seal. The gripping element may be at a position corresponding to an end of an opening formed in the pouch. The gripping element may be positioned on the same side of the pouch as the tear start. Thus, the gripping element is positioned such that it does not hinder removal of the catheter/sleeve from the pouch as it is located in a position that is directly accessible through an opening formed adjacent to the upper edge of the pouch. The position of the gripping element on a lateral edge of the pouch is also beneficial when the gripping element engages the catheter as described below as it can help to ensure the catheter passes through the wetting region when the wetting region is adjacent the base of the pouch.

The gripping element may detach from the sleeve when a threshold tension is applied on the sleeve in a direction away from the gripping element. The threshold tension may be at least 5 N, 10 N, 15 N, 20 N or 30 N. The threshold tension may be no more than 40 N, 30 N, 20 N, 15 N or 10 N. Preferably, the threshold tension is 20 N. Thus, the sleeve/catheter is not accidentally detached from the gripping element and requires deliberate, but not excessive, force applied by the user.

The gripping element may engage the sleeve in any suitable way, for example through an interference fit or breakable seal. The gripping element may comprise a temporary attachment between the sleeve and the pouch, for example a spot weld. The gripping element may comprise a narrow channel in the pouch which restricts movement of the sleeve through it by friction. The pouch may comprise a join between the two walls of the pouch which together with a lateral edge of the pouch defines the narrow channel. The gripping element may comprise an aperture, for example the gripping element may be an O-ring. The gripping element may be configured to engage the handling element. The aperture may have a diameter that is smaller than an outer diameter of the handling element. The catheter may pass through the aperture. The sleeve may not pass through the aperture. The sleeve may be push-fit into the aperture. Thus, the sleeve can be unfurled and then detached from the sleeve in a simple manner.

The gripping element may be configured to engage the catheter. The gripping element may be configured to direct the catheter through the wetting region during withdrawal of the catheter from the pouch.

Consequently, according to a third aspect of the present invention there is provided a packaged catheter assembly comprising a catheter having a proximal end for insertion into the body and a distal end, a pouch comprising a gripping element configured to engage the catheter, and a fluid reservoir configured to release wetting fluid into the pouch so as to wet the catheter, wherein: the catheter and fluid reservoir are arranged within the pouch; the pouch is configured to provide an opening through which the catheter may be withdrawn; the pouch comprises a wetting region in which wetting fluid preferentially collects; the wetting region is distal the opening; and the gripping element is configured to direct the catheter through the wetting region during withdrawal of the catheter from the pouch.

Thus, the gripping element guides the catheter through the wetting region during withdrawal of the catheter from the pouch to ensure the catheter is adequately wetted and ready for use.

The catheter may be configured to be drawn through the gripping element during withdrawal of the catheter from the pouch. The gripping element may restrict the direction of an axis of the catheter. The axis of the catheter may be restricted to within 30, 20, 10 or 5 degrees of an axis of the gripping element. The axis of the catheter may be restricted by the handling element. The axis of the gripping element may be directed towards the wetting region. The gripping element may comprise an aperture through which the catheter may pass, for example the gripping element may be an O-ring attached to the pouch, or may be a section of the sleeve attached to the pouch, or may be a narrow channel formed in the pouch as described above. Thus, the gripping element, or more particularly its aperture may ensure the catheter is angled towards the wetting region as it passes through the aperture, this can help to ensure the catheter passes through the wetting region.

The gripping element may engage the sleeve and/or the catheter. Thus, the catheter can be guided through the wetting region either indirectly via the sleeve or directly, for example in situations where the catheter assembly does not comprise a sleeve.

The gripping element may be located at a position outside the wetting region. The catheter may be configured such that during withdrawal of the catheter from the pouch it passes through the wetting region before it reaches the gripping element. The catheter may be configured to be withdrawn from the pouch in a direction parallel to the axis of the gripping element, or more particularly its aperture. Thus, the gripping element can be positioned such that it does not hinder withdrawal of the catheter or block wetting fluid from reaching the catheter in the wetting region.

The catheter may be resiliently biased into a straight configuration. The catheter may have a threshold rigidity to ensure that it passes through the wetting region. The threshold rigidity may ensure that the tangent of the catheter resists changing by more than 10, 20, 30, 40, 50, 60, 70 or 80 degrees over a length equivalent to the distance between the gripping element and wetting region. Thus, the catheter is urged into the wetting region by the rigidity of the catheter and the action of the gripping element.

The wetting region may be configured to collect a pool of wetting fluid, for example as described in relation to the first aspect above.

As will be appreciated by the skilled person, a typical male urinary catheter has a length around 40-45 cm. Given the pouch may have a height of up to 25 cm as described above. The catheter may be longer than the pouch. Thus, the catheter may be curved or coiled to fit within the pouch and the gripping element ensures that the catheter passes through the wetting region.

Accordingly in one embodiment, there is provided a packaged catheter assembly comprising a catheter having a proximal end for insertion into the body and a distal end, a pouch comprising a gripping element configured to engage the catheter, and a fluid reservoir configured to release wetting fluid into the pouch so as to wet the catheter, wherein: the catheter and fluid reservoir are arranged within the pouch; the pouch is configured to provide an opening through which the catheter may be withdrawn; the pouch comprises a wetting region in which wetting fluid preferentially collects; the wetting region is distal the opening; and the gripping element is configured to direct the catheter through the wetting region during withdrawal of the catheter from the pouch, wherein the catheter is longer than the pouch.

As mentioned above, the wetting region may be adjacent to the base of the pouch whereas the gripping element may be positioned in an upper half of the pouch. The catheter may therefore be curved between the gripping element and the wetting region. Thus, the catheter assembly can be arranged so that the catheter follows a curved path as it is withdrawn. This helps to minimise the physical size of the pouch.

Accordingly in one embodiment, there is provided a packaged catheter assembly comprising a catheter having a proximal end for insertion into the body and a distal end, a pouch comprising a gripping element configured to engage the catheter, and a fluid reservoir configured to release wetting fluid into the pouch so as to wet the catheter, wherein: the catheter and fluid reservoir are arranged within the pouch; the pouch is configured to provide an opening through which the catheter may be withdrawn; the pouch comprises a wetting region in which wetting fluid preferentially collects; the wetting region is distal the opening; and the gripping element is configured to direct the catheter through the wetting region during withdrawal of the catheter from the pouch, wherein the catheter is curved between the gripping element and the wetting region.

The packaged catheter assembly may comprise a fluid collection bag. The fluid collection bag may be arranged within the pouch. The pouch may comprise an interaction region (as described above) to form an opening in the pouch and allow the catheter and fluid collection bag to be removed from the pouch. The fluid collection bag may be arranged adjacent to the opening.

Thus, according to a fourth aspect of the present invention there is provided a packaged catheter assembly comprising a catheter, a pouch, and a fluid collection bag configured to receive fluid from the catheter in use, wherein: the catheter and fluid collection bag are arranged within the pouch; the pouch comprises an interaction region to form an opening in the pouch and allow the fluid collection bag and catheter to be removed from the pouch; and the fluid collection bag is arranged adjacent to the opening.

With this arrangement, the fluid collection bag protects the catheter from coming into contact with dirt as the pouch is opened and also helps prevent the user from accidentally touching the catheter during opening of the pouch. This helps to reduce risk of infection during use of the pouch as the catheter is less likely to be dirty.

In addition, where the pouch must be shaken to distribute wetting fluid within it, advantageously the fluid collection bag also reduces the amount of fluid which reaches the top of the bag and does not otherwise act to wet the surface of the catheter.

The fluid collection bag may be any suitable shape or size, for example rectangular, circular, elliptical, cuboid, spherical, etc. The fluid collection bag may comprise a front panel and a rear panel. The fluid collection bag may comprise a peripheral bond joining the periphery of the front panel and rear panel to form the fluid collection bag. The peripheral bond may define a base, two lateral edges and an upper edge of the fluid collection bag. The two lateral edges may comprise a right lateral edge and a left lateral edge. The right lateral edge and left lateral edge may be defined as the right and left sides of the bag when viewing the bag with the rear panel behind the front panel, the base at the bottom of the bag and the upper edge at the top of the bag. The bag may have a width between the left lateral edge and right lateral edge of at least 10, 12, 15 or 20 cm. The bag may have a width no more than 25, 20, or 15 cm. Preferably, the width is between 10 and 20 cm, most preferably between 12 and 15 cm. The base may define a bottom of the bag. The upper edge may define a top of the bag. The height of the bag from the base to the upper edge may be at least 25, 30, 35 or 40 cm. The height of the bag may be no more than 45, 40, 35 or 30 cm. The height may be between 25 and 40 cm, 30 and 35 cm, or most preferably 33 and 35 cm. The bag may be configured to hold at least 500, 700, or 1000 ml of fluid, and may hold no more than 1000 ml, for example, it may hold at least 700 ml of fluid.

The fluid collection bag may comprise a flexible plastics material. For example, polypropylene (PP), polyethylene terephthalate (PET), low density polyethylene (LDPE), metalized polyester (MET PET), orientated polypropylene (OPP), or polyvinyl chloride (PVC).

The peripheral bond may provide a water-tight seal. Preferably, the peripheral bond provides a sterile seal. The peripheral bond may comprise any one or more of: a weld; mechanical seal; heat seal; pressure seal; adhesive; solvent bond; ultraviolet bond; ultrasonic weld; laser weld; impulse weld; or friction weld. This ensures that the catheter is maintained within a sterile environment prior to use and also that once the catheter has been used any fluid contained within the bag does not leak out unintentionally.

The fluid collection bag may comprise an inlet to allow fluid to enter the bag from the distal end of the catheter and optionally via the sleeve. The inlet may be connected to one end of the sleeve which corresponds to the distal end of the catheter. The inlet may comprise a one-way valve which prevents fluid flow through the inlet from the fluid collection bag to the sleeve and/or catheter. Thus, the inlet allows liquid to pass into the fluid collection bag but optionally may prevent fluid flow out of the bag and into the sleeve. This can reduce the likelihood of leaking.

The fluid collection bag may be configured to be withdrawn through the opening before the catheter. The fluid collection bag may be configured to withdraw the catheter from the pouch. Thus, the fluid collection bag facilitates removal of the catheter from the pouch without the user having to touch the catheter.

The fluid collection bag may be arranged to span across a majority of the width of the pouch. The fluid collection bag may span at least 50%, 60%, 70%, 80%, or 90% of the width of the pouch. Thus, the fluid collection bag forms a barrier across the opening in the pouch and protects the contents of the pouch.

The fluid collection bag may obscure the catheter from the opening. The fluid collection bag may obscure the rest of the contents of the bag from the opening. Thus, the user is less likely to see and then immediately grasp or touch the catheter itself.

The fluid collection bag may be configured to separate the two walls of the pouch. The fluid collection bag may be configured to separate the two walls of the pouch at the opening of the pouch. Thus, once the opening is formed in the pouch, the fluid collection bag removes the need for the user to separate the walls of the pouch which can be inconvenient or difficult, especially for users with reduced mobility.

The inlet of the fluid collection bag may be arranged adjacent to one lateral edge of the pouch. Where the packaged catheter assembly comprises a gripping element and/or a tear start, the inlet of the fluid collection bag may be arranged on the same side of the pouch as the gripping element and/or tear start. Thus, the inlet is positioned to ensure the distal end of the catheter is arranged along the same side of the pouch as the gripping element and/or tear start. This helps the catheter to be withdrawn in a smooth and straight pulling action parallel to the lateral edge of the bag which reduces the force required to remove the catheter from the pouch.

The catheter may be arranged within the pouch in a curved and/or coiled configuration. The distal end of the catheter may be adjacent to the inner (left or right) lateral edge near the upper edge of the pouch. The catheter may extend down the inside of the pouch. The catheter may extend down through the aperture in the gripping element. The catheter may have a broadly elliptical anti-clockwise (or clockwise) coil as viewed with left lateral edge on the left, and upper edge at the top. The coil may have a single turn around the inner perimeter of the left lateral edge (or right lateral edge), base, and right lateral edge (or left lateral edge) of the pouch. The proximal end of the catheter may rest on the outside of the catheter's coil. The proximal end of the catheter may rest adjacent to the left lateral edge (or right lateral edge and base of the pouch. Thus, the catheter is safely stored within the pouch in a curved configuration which minimises risk of damage to the catheter—for example through kinking of the catheter—and also facilitates easy withdrawal of the catheter by pulling it by its distal end.

Accordingly in one embodiment there is provided a packaged catheter assembly comprising a catheter having a proximal end for insertion into the body and a distal end, a pouch comprising a gripping element configured to engage the catheter, and a fluid reservoir configured to release wetting fluid into the pouch so as to wet the catheter, wherein: the catheter and fluid reservoir are arranged within the pouch; the pouch is configured to provide an opening through which the catheter may be withdrawn; the pouch comprises a wetting region in which wetting fluid preferentially collects; the wetting region is distal the opening; and the gripping element is configured to direct the catheter through the wetting region during withdrawal of the catheter from the pouch, wherein the catheter is arranged within the pouch in a coiled configuration.

The inlet of the fluid collection bag may be positioned at a corner of the peripheral bond. The fluid collection bag may comprise a neck adjacent to the inlet. The neck may comprise one or more projections extending into the fluid collection bag. The one or more projections may comprise regions of the peripheral bond that extend into the bag. The one or more projections may restrict fluid flow through a region of the bag adjacent to the inlet. The projections may be configured to provide gripping points adjacent to the inlet. Thus, the fluid collection bag resists the flow of fluid back towards the inlet and also is more easily handled by the user.

Any one or more of the one or more projections may have any one or more of the features of a projection as described below.

A projection may have any suitable size or shape. A projection may be triangular, square, rectangular, circular, ellipsoidal or irregularly shaped, preferably, a projection is triangular. A point on the projection furthest from an edge of the bag may be a (right-angled) corner in the projection. The projection may be a right-angled triangle. The hypotenuse of the projection may be aligned parallel to an edge of the bag. Thus, the projections can be suitably shaped to minimise loss of volume in the bag due to the projections. In addition, its shape can help to direct fluid flow within the bag, for example assisting flow into the bag and reducing flow out of the bag.

A projection may extend along no more than 90%, 80%, 70%, 60%, 50%, 40%, 30%, 20% or 10% of the length of an edge of the bag. A projection may extend by at least 10%, 20%, 30%, 40%, 50%, 60%, 70%, 80%, or 90% of the length of an edge of the bag. A projection may comprise a corner of the bag. A projection may not comprise from a corner of the bag. Thus, the projections can be flexibly sized and positioned to be most effective.

A projection may be a lateral projection extending from a left (or right) lateral edge of the bag. The lateral projection may be provided on the same side of the bag as the inlet. The lateral projection may extend along 10-40% of the length of the lateral edge of the bag, preferably 25%. The lateral projection may extend across 5-20% of the width of the bag, preferably 10%. The lateral projection may be a right-angled triangle. The right-angled vertex of the lateral projection may project into the bag. The right-angled vertex may be positioned below the inlet inside the bag. The right-angled vertex may be positioned 10% of the way from the upper edge to the base of the bag. The non-right-angled vertices of the lateral projection may be provided on the lateral edge of the bag from which the lateral projection extends. Thus, the lateral projection can assist in directing the flow of fluid into the bag and also helps prevent fluid flowing directly from the bag back up into the sleeve/catheter.

A projection may be an end projection extending from the upper edge or base of the bag. Preferably, the end projection extends from the upper edge. The end projection may be provided adjacent to the inlet. The end projection may extend along 40-95% of the width of the bag, preferably 90%. The end projection may extend from a corner of the bag. The end projection may extend down 5-20% of the length of the bag, preferably 10%. The end projection may be a right-angled triangle. The right-angled vertex of the end projection may project into the bag. The right-angled vertex may be positioned adjacent the inlet inside the bag. The right-angled vertex may be positioned 20% of the way across the width of the bag. The non-right-angled vertices of the lateral projection may be provided on the edge of the bag from which the end projection extends. Thus, the end projection can assist in directing the flow of fluid into the bag and also helps prevent fluid flowing directly from the bag back up into the sleeve/catheter.

In a preferred embodiment, the fluid collection bag comprises both a lateral projection and an end projection extending from the upper edge of the bag as described above. Thus, the two projections work together to assist the user in handling the bag and also to manage fluid flow into/out of the bag.

A projection may comprise a rib. The rib may resist bending or deformation in a given direction. The rib may facilitate folding of the fluid collection bag. The rib may assist the user in grasping or gripping the bag. The strengthening element may comprise a region of the projection that is thicker or thinner than the rest of the projection. The rib may be any suitable shape or size, such as a line which could be straight or curved. The rib may extend across the projection. The rib may extend from an edge of the bag. The rib may extend from a midpoint of the projection along the edge of the bag. The rib may extend to a point on the projection furthest from the edge of the bag, for example a right-angled vertex of a right-angled projection. The rib may be at an angle (i.e. not parallel or perpendicular to) an edge of the bag. Preferably, the lateral projection may comprise a rib. Thus, the mechanical properties of the projection can be controlled through use of a rib to the benefit of the user.

A projection may be a join that is independent from the peripheral bond of the fluid collection bag. A join may have any one or more of the features of a projection as described herein. A join can be beneficial to reduce the total area of the panels of the fluid collection bag that are sealed together.

The fluid collection bag may comprise a finger hole. The finger hole may be sized to allow a user to put their finger through the finger hole and handle the bag. Thus, the finger hole allows the bag to be handled more easily by a user. The finger hole may be provided at an edge of the bag. The finger hole may be provided in the peripheral bond of the bag. The finger hole may be provided at a corner of the bag. The finger hole may be provided at any of the following corners: where the left lateral edge meets the base; where the base meets the right lateral edge; where the right lateral edge meets the upper edge; or where the upper edge meets the left lateral edge. Where the finger hole is provided in the bag, the peripheral bond may be shaped to accommodate the finger hole. The peripheral bond may truncate a corner of the bag to accommodate the finger hole. The peripheral bond may project into the bag to accommodate the finger hole. As an alternative to the finger hole, the fluid collection bag may comprise a series of ridges or otherwise easily gripped surface or feature in the place of the finger hole to allow the bag to be easily handled.

The fluid collection bag may comprise more than one finger hole, for example at least two, three or more finger holes. One, two, three or more, or each finger hole may have any one or more of the features of the finger hole described above. Thus, easy handling of the bag is facilitated through positioning of finger holes or equivalent features which also does not restrict or affect the volume of liquid that can be contained by the bag.

The finger hole may be provided in a projection of the fluid collection bag. Preferably, the end projection as described above comprises a finger hole. The finger hole may be positioned adjacent to the right-angled vertex of the end projection.

The one or more finger holes may be configured to allow the bag to be rotated to a horizontal position wherein the base of the bag is aligned vertically. In a preferred embodiment, the fluid collection bag comprises a finger hole at either end of the right lateral edge, and/or a finger hole at either end of the left lateral edge, and/or a finger hole along the upper edge and a finger hole along the base. Advantageously, this allows the bag to be rotated in a controlled manner which aids with handling and using the bag.

The fluid collection bag may comprise a fill level indicator. The fill level indicator may comprise fill markers. The fill markers may allow the user to measure the volume of fluid contained within the fluid collection bag. The fill markers may be regularly spaced or irregularly spaced. The fill markers may correspond to an absolute volume of liquid contained within the bag. The fill markers may correspond to a proportion of the volume of the bag which is filled with fluid. The fill markers may indicate when a safe fill level of the bag has been reached. The fill level indicator may be positioned in a transparent or translucent region of the bag. Thus, the fill level indicator ensures the bag is not overfilled.

The pouch may comprise a sheath arranged around the fluid collection bag. The sheath may be configured to inhibit wetting fluid from the fluid reservoir from wetting the exterior of the fluid collection bag.

According to a fifth aspect of the present invention there is provided a packaged catheter assembly comprising a pouch, a catheter, a fluid reservoir, and a fluid collection bag configured to receive fluid from the catheter, wherein: the catheter, fluid reservoir and fluid collection bag are arranged within the pouch; the fluid reservoir is configured to release wetting fluid into the pouch to wet the catheter; and the pouch comprises a sheath arranged around the fluid collection bag to inhibit wetting fluid from the fluid reservoir wetting the exterior of the fluid collection bag.

Thus, the sheath makes the fluid collection bag easier to use and more comfortable for the user to handle as it the sheath prevents the exterior of the fluid collection bag from becoming wet. When the exterior is wet, the bag may be slippery which can make handling more difficult, in addition, it can be more likely to stick to itself due to surface tension forces between two wet surfaces.

The fluid collection bag may be provided in a stowed configuration. The sheath may retain the fluid collection bag in the stowed configuration. In the stowed configuration, the fluid collection bag may be folded or rolled to reduce its size in any one dimension. The stowed configuration may be a folded or rolled configuration. Thus, the size of the fluid collection bag is minimised inside the pouch and this assists the user in removing the bag and other elements of the packaged catheter assembly from the pouch. The stowed configuration of the fluid collection bag further inhibits fluid flow onto the exterior surface of the fluid collection bag as it must penetrate between folds.

The sheath may be any suitable shape or size. The sheath is preferably tubular. The sheath may have one closed end. The sheath may enclose the majority of the (stowed) fluid collection bag. The sheath may enclose at least 50%, 60%, 70%, 80% or 90% of the outer surface of the (stowed) fluid collection bag, preferably at least 66%. Thus, the sheath is easy to manufacture and effectively inhibits wetting of the majority of the exterior of the fluid collection bag.

The sheath may comprise a fluid (or liquid) impermeable material. For example, the sheath may be formed of a thermoplastic polyurethane (TPU) or low-density polyethylene (LDPE).

The pouch may comprise an interaction region allowing the user to create an opening in the pouch. The axis of the sheath may be arranged parallel to the tear line of the pouch and/or the opening formed in the pouch. This helps to protect the contents of the pouch from dirt when opening the pouch as the sheath provides an additional fluid impermeable barrier.

A first end (for example the closed end) of the sheath may be arranged adjacent to one lateral edge of the pouch, or a first end of the opening. A second end (for example an open end opposite the closed end) of the sheath may be arranged adjacent a second end of the opening. For example, a first end, such as the closed end may be on the opposite side of the pouch as the inlet of the fluid collection bag/gripping element and/or tear start. Thus, the sheath and fluid collection bag are positioned to ensure the distal end of the catheter is arranged along the same side of the pouch as the gripping element and/or tear start. This helps the catheter to be withdrawn in a smooth and straight pulling action parallel to the lateral edge of the bag which reduces the force required to remove the catheter from the pouch.

The sheath may be configured to be removed from the fluid collection bag. The sheath may be configured to be removed from the fluid collection bag after withdrawal of the fluid collection bag from the pouch. Thus, the sheath ensures the fluid collection bag is protected while in the pouch but easily useable when required.

The packaged catheter assemblies of the first to fifth aspects may include any one or more features of a packaged catheter assembly as defined in broad terms, or according to any other of the first to fifth aspects set out above. The packaged catheter assemblies of the first to fifth aspects may comprise any of the optional features of the others of the first to fifth aspects without necessarily including all the features required of them. That is to say, an optional feature which happens to be set out following one particular aspect does not necessarily apply only to that aspect, so, for example, the disclosure provides for the packaged catheter assembly of the fifth aspect comprising a pouch, a catheter, a fluid reservoir, and a fluid collection bag configured to receive fluid from the catheter, wherein: the catheter, fluid reservoir and fluid collection bag are arranged within the pouch; the fluid reservoir is configured to release wetting fluid into the pouch to wet the catheter; and the pouch comprises a sheath arranged around the fluid collection bag to inhibit wetting fluid from the fluid reservoir wetting the exterior of the fluid collection bag, wherein the fluid collection bag further comprises an inlet and one or more projections as described in relation to the fourth aspect.

According to a sixth aspect of the present invention there is provided a method manufacturing a packaged catheter assembly comprising the steps of providing a pouch comprising an absorbent material, and arranging a catheter within the pouch.

The method of the sixth aspect of the invention may be a method of forming the packaged catheter assembly of the first aspect of the invention, which, of course, may include any optional feature outlined above.

The method may comprise positioning the absorbent material adjacent to a mouth, opening or tear line of the pouch.

According to a seventh aspect of the present invention, there is provided a method manufacturing a packaged catheter assembly comprising the steps of providing a catheter having a proximal end for insertion into the body and a distal end, a sleeve enclosing the catheter, a pouch comprising a gripping element configured to engage the sleeve, and a fluid reservoir configured to release wetting fluid into the pouch so as to wet the catheter, and arranging the catheter and fluid reservoir within the pouch; wherein the gripping element is configured to unfurl the sleeve along the length of the catheter and detach from the sleeve.

The method of the seventh aspect of the invention may be a method of forming the packaged catheter assembly of the second aspect of the invention, which, of course, may include any optional feature outlined above.

The method may comprise providing the sleeve in a furled configuration. The method may comprise attaching the sleeve to the gripping element.

The method may comprise providing two walls and sealing them around their periphery to form a peripheral seal defining the pouch. The gripping element may be attached to the pouch by the peripheral seal. The gripping element may be attached to the pouch during formation of the peripheral seal. The gripping element may be formed during formation of the peripheral seal. The method may comprise sealing the sleeve to the pouch to form the gripping element. The method may comprise joining the two walls of the pouch together (at a location independent of the peripheral seal) to form the gripping element. Thus, the gripping element is easily and securely integrated into the pouch.

According to an eighth aspect of the present invention there is provided a method of forming a packaged catheter assembly comprising the steps of providing a catheter having a proximal end for insertion into the body and a distal end, a pouch comprising a gripping element configured to engage the catheter, and a fluid reservoir configured to release wetting fluid into the pouch so as to wet the catheter, and arranging the catheter and fluid reservoir within the pouch, wherein the pouch is configured to provide an opening through which the catheter may be withdrawn, the pouch comprises a wetting region in which wetting fluid preferentially collects, the wetting region is distal the opening, and the gripping element is configured to direct the catheter through the wetting region during withdrawal of the catheter from the pouch.

The method of the eighth aspect of the invention may be a method of forming the packaged catheter assembly of the third aspect of the invention, which, of course, may include any optional feature outlined above.

According to a ninth aspect of the present invention there is provided a method of manufacturing a packaged catheter assembly, the method comprising the steps of providing a catheter, a pouch, and a fluid collection bag configured to receive fluid from the catheter in use, arranging the catheter and fluid collection bag within the pouch wherein the pouch comprises an interaction region to form an opening in the pouch and allow the fluid collection bag and catheter to be removed from the pouch, and the fluid collection bag is arranged adjacent to the opening.

The method of the ninth aspect of the invention may be a method of manufacturing the packaged catheter assembly of the fourth aspect of the invention, which, of course, may include any optional feature outlined above.

The method may comprise arranging the catheter in the pouch and then arranging the fluid collection bag in the pouch. This can ensure the fluid collection bag remains adjacent to the opening.

The method may comprise attaching the catheter to an inlet of the fluid collection bag. The method may comprise arranging the inlet on one side of the fluid collection bag.

According to a tenth aspect of the present invention there is provided a method manufacturing a packaged catheter assembly comprising the steps of providing a pouch, a catheter, a fluid reservoir, and a fluid collection bag configured to receive fluid from the catheter, arranging the catheter, fluid reservoir and fluid collection bag within the pouch, and providing a sheath arranged around the fluid collection bag; wherein the fluid reservoir is configured to release wetting fluid into the pouch to wet the catheter, and the sheath is configured to inhibit wetting fluid from the fluid reservoir wetting the exterior of the fluid collection bag.

The method of the tenth aspect of the invention may be a method of manufacturing the packaged catheter assembly of the fifth aspect of the invention, which, of course, may include any optional feature outlined above.

The method may comprise folding or rolling the fluid collection bag. The fluid collection bag may be folded or rolled before it is arranged inside the pouch. The method may comprise arranging the sheath around the folded or rolled fluid collection bag. The sheath may be arranged around the folded or rolled fluid collection bag before it is arranged in the pouch. The method may comprise attaching the catheter to the fluid collection bag. The catheter may be attached to the fluid collection bag before it is arranged in the pouch. The method may comprise arranging the sheath and/or catheter with the fluid collection bag in the pouch simultaneously.

According to an eleventh aspect of the present invention there is provided a method of unpackaging a packaged catheter assembly, the packaged catheter assembly comprising a pouch and a catheter, wherein the pouch is configured to contain the catheter within it and wherein the pouch comprises an absorbent material, the method comprising removing the catheter from the pouch.

The method of the eleventh aspect of the invention may be a method of unpackaging a packaged catheter assembly of the first aspect of the invention, which, of course, may include any optional feature outlined above and may be manufactured according to the sixth aspect of the invention.

The method may comprise wetting the catheter and absorbing excess wetting fluid with the absorbent material.

The method may comprise introducing the catheter by its proximal end into the urethra. The method may comprise allowing fluid to pass out of the body via the catheter. Consequently, the method may be a method of using a packaged catheter assembly.

The method may comprise absorbing liquid that has passed through the catheter. Thus, the volume of free liquid is reduced by the absorbent material and this can make transportation and disposal of the liquid easier and more convenient for the user.

The method comprising absorbing liquid present on the outside of the catheter. This may preferably be done after use of the catheter. The method may comprise placing the catheter within the pouch. The method may comprise closing the pouch. Thus, any excess liquids are absorbed by the absorbent material and retained within the pouch after use of the catheter.

According to a twelfth aspect of the present invention, there is provided a method of unpackaging a packaged catheter assembly, the packaged catheter assembly comprising a pouch comprising a gripping element, a catheter contained within the pouch and a sleeve enclosing the catheter, the method comprising the steps of releasing wetting fluid from the fluid reservoir into the pouch so as to wet the catheter, unfurling the sleeve along the length of the catheter using the gripping element which is configured to engage the sleeve, and detaching the sleeve from the gripping element.

The method of the twelfth aspect of the invention may be a method of unpackaging a packaged catheter assembly of the second aspect of the invention, which, of course, may include any optional feature outlined above and may be manufactured according to the seventh aspect of the invention.

The method may comprise forming an opening in the pouch. The method may comprise withdrawing the catheter through the opening. The method may comprise allowing the gripping element to remain in the pouch. The method may comprise unfurling the sleeve by withdrawing the catheter from the pouch. Thus, the sleeve is automatically unfurled as the catheter is withdrawn from the pouch.

The method may comprise rupturing/opening/bursting the fluid reservoir within the pouch. The method may comprise locating the fluid reservoir within the (closed/sealed) pouch. The method may comprise locating the fluid reservoir via a window in the pouch and/or a wetting marker disposed on the pouch. Thus, wetting fluid is easily released into the pouch.

The step of forming an opening may comprise using an interaction region of the pouch to form the opening. The method may comprise grasping a tear-away region of the pouch. The method may comprise grasping an aperture in the interaction region/tear-away region. The method may comprise tearing the pouch from a tear start. The method may comprise tearing the pouch along a tear line. The method may comprise tearing the pouch until a tear stop. Thus, the pouch is easily opened by the user.

The method may comprise removing moisture or liquids from the exterior of the sleeve using an absorbent material disposed within the pouch. Thus, the method of the twelfth aspect may be combined with features of the eleventh aspect (or vice versa) to provide a sleeve with an exterior that is dry and easy to handle.

The method may comprise handling the catheter via the unfurled sleeve. The method may comprise introducing the catheter by its proximal end into the urethra. The method may comprise allowing fluid to pass out of the body via the catheter. Consequently, the method may be a method of using a packaged catheter assembly.

According to an thirteenth aspect of the present invention there is provided a method of unpackaging a packaged catheter assembly, the packaged catheter assembly comprising a catheter having a proximal end for insertion into the body and a distal end, a pouch comprising a gripping element configured to engage the catheter, and a fluid reservoir configured to release wetting fluid into the pouch so as to wet the catheter, wherein the catheter and fluid reservoir are arranged within the pouch, the method comprising the steps of releasing wetting fluid into the pouch so as to wet the catheter, allowing wetting fluid to collect in a wetting region of the pouch, and withdrawing the catheter from the pouch, wherein the gripping element is configured to direct the catheter through the wetting region during withdrawal of the catheter from the pouch.

The method of the thirteenth aspect of the invention may be a method of unpackaging a packaged catheter assembly of the third aspect of the invention, which, of course, may include any optional feature outlined above and may be manufactured according to the eighth aspect of the invention.

The method may comprise restricting the direction of an axis of the catheter. The method may comprise passing the catheter through the wetting region before it reaches the gripping element.

The method may comprise introducing the catheter by its proximal end into the urethra. The method may comprise allowing fluid to pass out of the body via the catheter. Consequently, the method may be a method of using a packaged catheter assembly.

According to a fourteenth aspect of the present invention there is provided a method of unpackaging a packaged catheter assembly, the packaged catheter assembly comprising a catheter, a fluid collection bag configured to receive fluid from the catheter in use, and a pouch containing the catheter and fluid collection bag, the method comprising using an interaction region to form an opening in the pouch to allow the catheter and fluid collection bag to be removed from the pouch, wherein the fluid collection bag is arranged adjacent to the opening.

The method of the fourteenth aspect of the invention may be a method of unpackaging a packaged catheter assembly according to the fourth aspect of the invention and may, of course, include any optional feature outlined above and the packaged catheter assembly may be manufactured according to the ninth aspect of the invention.

The method may comprise withdrawing the fluid collection bag from the pouch before the catheter. The method may comprise withdrawing the catheter from the pouch using the fluid collection bag.

The method may comprise unfolding the fluid collection bag. The method may comprise handling the fluid collection bag via one or more projections and/or one or more finger holes disposed on the fluid collection bag.

The method may comprise measuring the fluid contained in the fluid collection bag via a fill level indicator disposed on the fluid collection bag.

The method may comprise introducing the catheter by its proximal end into the urethra. The method may comprise allowing fluid to pass out of the body via the catheter. Consequently, the method may be a method of using a packaged catheter assembly.

According to a fifteenth aspect of the present invention there is provided a method of unpackaging a packaged catheter assembly, the packaged catheter assembly comprising a catheter, a fluid reservoir, and a fluid collection bag configured to receive fluid from the catheter, and a pouch containing the catheter, fluid reservoir and fluid collection bag, the method comprising releasing wetting fluid into the pouch so as to wet the catheter, wherein the pouch comprises a sheath arranged around the fluid collection bag to inhibit wetting fluid from the fluid reservoir wetting the exterior of the fluid collection bag.

The method of the fifteenth aspect of the invention may be a method of unpackaging a packaged catheter assembly according to the fifth aspect of the invention and may, of course, include any optional feature outlined above and the packaged catheter assembly may be manufactured according to the tenth aspect of the invention.

The method may comprise withdrawing the fluid collection bag from the pouch using the sheath. The method may comprise removing the sheath from the fluid collection bag, which may optionally be done after withdrawal of the fluid collection bag from the pouch. The method may comprise unfolding the fluid collection bag, after removal of the sheath from the fluid collection bag.

The method may comprise introducing the catheter by its proximal end into the urethra. The method may comprise allowing fluid to pass out of the body via the catheter. Consequently, the method may be a method of using a packaged catheter assembly.

The methods of the sixth to fifteenth aspects of the present invention may of course individually include any one or more of the features, optional or otherwise, of one another and further may include any one or more of the features, optional or otherwise, of the first to fifth aspects of the present invention.

In any aspect, the catheter assembly is preferably a closed catheter assembly, that is that the catheter assembly comprises a catheter coupled to a fluid collection bag, such that it is configured to collect and retain fluid released from the body through the catheter. The fluid collected/catheter assembly may then be disposed of by the user when is convenient. Of course, embodiments of the invention may also be open catheter assemblies in that fluid is not collected or retained by the catheter assembly. Likewise in any aspect the catheter is preferably a urinary catheter; and/or is an intermittent catheter. Thus, the features of aspects of the present invention allow intermittent male urinary catheters to be adequately wetted prior to use which can be more difficult than for other types of catheter which are generally shorter.

### DETAILED DESCRIPTION OF THE INVENTION

In order that the invention may be more clearly understood one or more embodiments thereof will now be described, by way of example only, with reference to the accompanying drawings, of which:

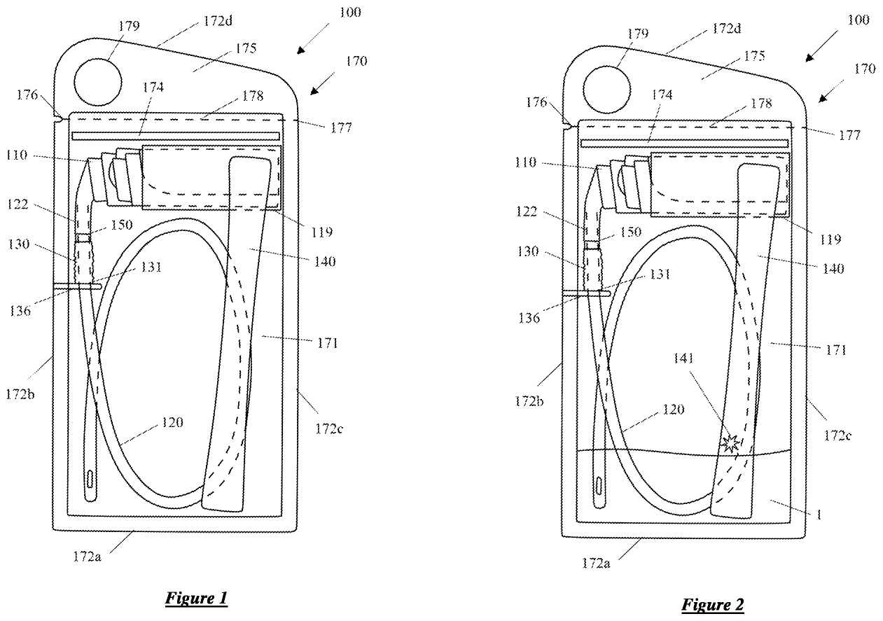

FIG. 1 is a cut-away front view of a first embodiment of a packaged catheter assembly;

FIG. 2 is a cut-away front view of the packaged catheter assembly of FIG. 1 where wetting fluid from the fluid reservoir has been released into the pouch;

FIG. 3 is a cut-away front view of the packaged catheter assembly of FIG. 1 during forming an opening in the pouch;

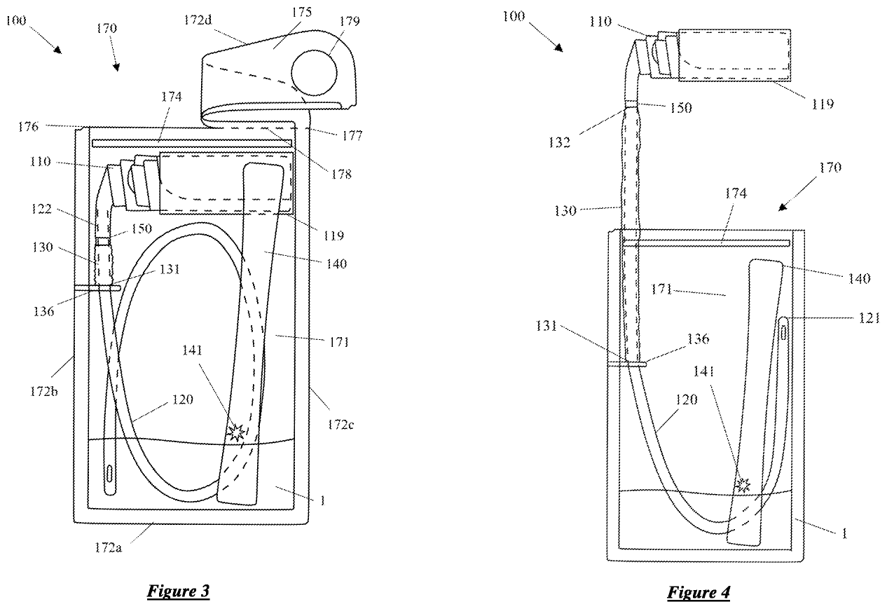

FIG. 4 is a cut-away front view of the packaged catheter assembly of FIG. 1 during withdrawal of the catheter assembly from the pouch;

FIG. 5 is a cut-away front view of the packaged catheter assembly of FIG. 1 during withdrawal of the catheter assembly from the pouch;

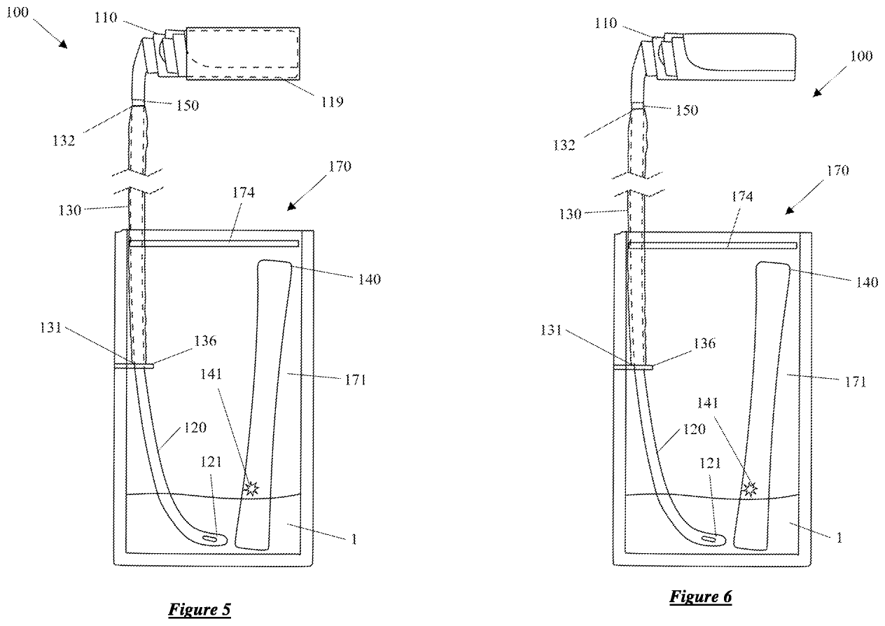

FIG. 6 is a cut-away front view of the packaged catheter assembly of FIG. 1 during withdrawal of the catheter assembly from the pouch; and

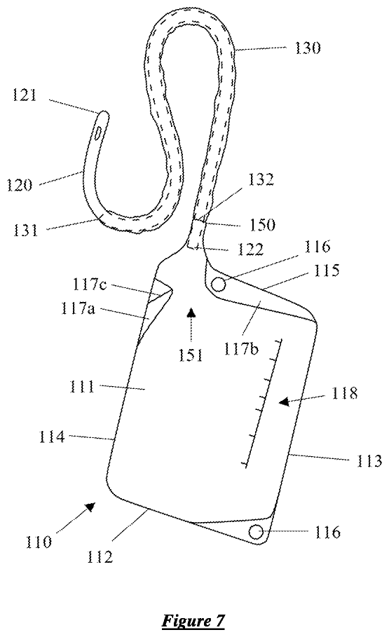

FIG. 7 is a cut-away front view of the catheter assembly of FIG. 1 where the closed catheter assembly has been removed from the pouch.

In the Figures, as is conventional, broken lines show hidden features.

Referring to FIGS. 1-7, an embodiment of a packaged catheter assembly 100 is shown.

The packaged catheter assembly 100 comprises a pouch 170 containing a fluid collection bag 110, a catheter tube, or “catheter” 120, a sleeve 130, and a fluid reservoir 140 comprising wetting fluid. The catheter 120 has a proximal end 121 for insertion into the body and a distal end 122, and the fluid collection bag 110 is attached to the distal end 122 of the catheter 120 and is arranged to receive fluid from the distal end 122 of the catheter 120, thereby forming a so-called “closed catheter assembly”.

The sleeve 130 of this embodiment is sized to enclose the catheter 120 along the majority of the length of the catheter 120. The sleeve 130 comprises a proximal end 131 corresponding to the proximal end 121 of the catheter 120 and a distal end 132 corresponding to the distal end 122 of the catheter 120. The distal end 132 of the sleeve 130 is attached to an inlet 150 of the fluid collection bag 110. The proximal end 131 of the sleeve comprises an annular handling element 133. The handling element 133 comprises a relatively rigid material compared to the sleeve 130 and facilitates movement of the proximal end 131 of the sleeve 130 up and down the catheter 120.

In this embodiment, the catheter 120 is a male urinary catheter made from a hydrophilic thermoplastic elastomer (TPE). The sleeve 130 of this example is a thermoplastic polyurethane (TPU) or low-density polyethylene (LDPE). Obviously those skilled in the art will be able to select suitable alternative materials.

The pouch 170 is formed from a front wall (not shown-cut away) and rear wall 171 of identical shape and size, and a peripheral seal joining the periphery of the walls to form the pouch 170 and containing its contents (i.e. the closed catheter assembly and fluid reservoir 140).

The peripheral seal defines a base 172a, a left lateral edge 172b, a right lateral edge 172c and an upper edge 172d of the pouch 170. The left lateral edge 172b and right lateral edge 172c being defined as the left and right sides of the pouch 170 when viewing the pouch 170 with the rear wall 171 behind the front wall, the base 172a at the bottom of the pouch 170 and the upper edge 172d at the top of the pouch 170. The peripheral seal thus defines a pouch 170 that is generally rectangular and suitably has a width between the lateral edges of between 60-110 mm, preferably 90-110 mm, for example about 100 mm, and a height from the base 172a to the upper edge 172d of between 100 to 250 mm, preferably 120 to 140 mm, for example about 130 mm. The base 172a defines the bottom of the pouch 170 in use, and the upper edge 172d the top. However, in other embodiments alternative shapes and sizes could be conceived, such as elliptical or circular walls forming correspondingly shaped pouches, or even a tubular pouch or pouch with more than two walls, e.g. a prism or other polygon.

The peripheral seal may be formed from any suitable bonding/sealing methods such as chemical adhesives, chemical bonding, ultrasonic bonding, or mechanical bonding. In alternative embodiments, the pouch 170 may be formed from a single piece of material. The pouch may then be formed by folding this single piece of material along a pouch fold line to form the walls. In addition, in some embodiments any number of folds may be used to form the pouch 170. For example, none of the edges may comprise a fold, one edge may comprise a fold (as in the illustrated example), or two or more of the edges may comprise a fold.

In this embodiment, the pouch 170 is formed from an opaque material and as such provides a discrete package for its contents which can make the user more comfortable in carrying the packaged catheter assembly 100 in day-to-day life. In addition, the exterior of the pouch 170 may have a textured or printed appearance, for example a matte appearance, and feel to reduce the impression that it is a medical device.

In this embodiment, the pouch 170 also comprises an interaction region. The interaction region forms the top part of the pouch 170 and spans between the right lateral edge 172c and left lateral edge 172b. The interaction region is used to provide access to the pouch through/near to the upper edge 172d. In this embodiment, the interaction region comprises a tapered tear-away region 175. The tear-away region 175 comprises a tear start 176 at the point the left lateral edge 172b meets the tear-away region 175, a tear stop 177 at a corresponding point on the right lateral edge 172c, and a tear line 178 spanning between the tear start 176 and tear stop 177. The tear line 178 comprises a line of weakness in the walls of the pouch 170, which may be formed by any suitable means but in this embodiment is laser etched. The tear start 176 comprises a notch to provide an area of weakness in the peripheral seal. This allows the tear-away region 175 to be separated from the pouch 170 by tearing the walls apart from the tear start 176 to the tear stop 41 along the tear line 178. Thus, the tear is provided beneath the location of the upper edge 172d and thus the pouch 170 may be opened provided access to its contents. In other embodiments, the tear stop 177 may comprise a small aperture in the walls to prevent further tearing of the pouch 170.

In order to provide the tapered shape, the tear-away region 175 extends upwards on the left side approximately 50% more than it does on the right side. The left side of the tear-away region 175 comprises a circular aperture 179 sized to allow a finger to pass through and grip the tear-away region 175. To ensure the pouch 170 is fully sealed, the front wall and rear wall 171 are sealed to one another over the entire tear-away region 175. In other embodiments, other seals such as a zip-lock arrangement, or hook and hook, hook and loop (VELCRO®) or the like may be used to provide access to the pouch (or to reseal the pouch after opening), and of course, the sizes and shapes set out above are exemplary.

In this embodiment, the pouch 170 also comprises an absorbent strip 174. The absorbent strip 174 is a thin piece of absorbent material that is arranged close to, and slightly below, the tear line 178. The absorbent strip is arranged on the inside of the rear wall 171 in this embodiment, but in other embodiments may be arranged on the front wall or both the front and rear walls 171. The absorbent strip 174 spans the majority of the tear line 178 and is configured to absorb any fluids that come into contact with it. In other embodiments, the absorbent strip 174 may be an absorbent layer and may line the front wall and/or rear wall 172, or may be replaced by an absorbent that is not shaped like a strip or layer, such as an absorbent sachet. The absorbent strip may be formed, for example, from a sponge material, an absorbent polymer, or a super absorbent for example polyacrylic acid (but those skilled in the art will easily identify alternatives, in light of this disclosure).

In this embodiment, the left lateral edge 172b of the pouch 170 comprises a gripping element 136 which projects into the inside of the pouch 170 at a point approximately mid-way between the base 172a and upper edge 172d of the peripheral seal. In this embodiment, the gripping element 136 is configured to engage the proximal end 131 of the sleeve 130 and to restrict the direction of the tangent to the catheter 120 at the gripping element 136. Consequently, the gripping element 136 can both control the position of the sleeve 130 on the catheter 120 and also control the position of the catheter 120 within the pouch 170.

In this embodiment, the gripping element 136 comprises an O-ring with aperture (not shown) through which the catheter 120 is arranged. The O-ring of the gripping element 136 has an inner diameter that is slightly smaller than the outer diameter of the handling element 133, thus, the handling element 133 may be push-fit within the gripping element 136. In other embodiments, the gripping element 136 instead comprise a temporary/frangible attachment between the pouch wall(s) and sleeve 130, for example a spot weld. In further alternative embodiments, the gripping element may comprise a narrow channel in the pouch, providing an aperture between the pouch walls that restricts movement of the sleeve 130 by friction. The narrow channel may be formed by forming a join between the walls and independent from the peripheral seal to define the narrow channel between the join and the left 172b (or right 172c) lateral edge of the peripheral seal.

In this embodiment, the aperture of the gripping element 136 comprises an axis extending perpendicular to the aperture and parallel to the left lateral edge 172c of the pouch 170. The gripping element 136 is configured to restrict the tangent of the catheter 120 to within a certain degree (e.g. about 20 degrees) of the axis of the aperture at the gripping element, as it is pulled out of the top of the pouch 170. In this embodiment, this is achieved through the handling element 133 which is push-fit inside the gripping element 133 and provides a tubular path through which the catheter 120 must pass. In this embodiment, the pouch 170 comprises a wetting region adjacent to the base 172a of the pouch 170, as described below. The axis of the aperture, and the path of the catheter 120 through the gripping element 136 defined by the handling element 133, is therefore pointing towards the wetting region and so is the catheter 120.

In this embodiment, the catheter 120 is resiliently biased into a straight configuration to the extent that over a distance equivalent to the distance between the gripping element 136 and base 172a/wetting region, the catheter 120 resists being bent by more than about 20 degrees. As such, due to the restriction of the tangent of the catheter 120 imposed by the gripping element 136 and the resilience of the catheter 120 itself, the catheter 120 extends away from the gripping element 136 substantially parallel to the left lateral edge 172c of the pouch 170 and extends down into the wetting region and adjacent to the base 172a of the pouch 170.

The catheter 120 is arranged within the pouch 170 in a curved and coiled configuration, with the distal end 122 adjacent to the inner left lateral edge 172b near the upper edge 172d of the pouch 170. The catheter 120 extends down the inside of the pouch 170, through the aperture in the gripping element 136 and into a broadly elliptical anti-clockwise coil (as viewed with left lateral edge 172b on the left, and upper edge 172d at the top). The coil has a single turn around the inner perimeter of the left lateral edge 172b, base 172a, and right lateral edge 172c of the pouch, with the proximal end 121 of the catheter 120 resting on the outside of the catheter's coil, adjacent to the left lateral edge 172b and base 172a of the pouch 170. Of course, the catheter uncoils as it is pulled out of the pouch, as described below.

In this embodiment, the fluid collection bag 110 is provided in a stowed configuration, which in this embodiment is a folded configuration, within the pouch 170 and is retained in the folded configuration by a sheath 119 provided around the folded fluid collection bag 110. The sheath 119 is tubular with one closed end and encloses the majority of the folded fluid collection bag 110, in this embodiment it encloses approximately two thirds of the length of the folded bag 110 but in other embodiments it may be more or less. The sheath 119 comprises a fluid impermeable material and as such, resists fluids from contacting the fluid collection bag 110.

In this embodiment, the sheath 119 and folded fluid collection bag 110 are arranged within the pouch 170 just below the absorbent strip 174 and with the axis of the tubular sheath 119 approximately parallel to the absorbent strip 174. The closed end of the sheath 119 is positioned adjacent to the right lateral edge 172c of the pouch 170 and the inlet 150 of the bag 110 receives the distal end 122 of the catheter 120 adjacent to the left lateral edge 172b of the pouch 170. The fluid collection bag 110 is therefore arranged adjacent to the opening formed in the pouch 170 by the tear-away region 175 as described further below and spans between the left lateral edge 172b and right lateral edge 172c.

In this embodiment, prior to use, the sleeve 130 is arranged around the catheter 120 in a furled configuration between the inlet 150 and gripping element 136. The distal end 132 of the sleeve 130 is attached to the inlet 150 and the proximal end 131 of the sleeve 130 is engaged by the gripping element 136. Consequently, the sleeve 130 in the furled configuration only covers a minority of the length of the catheter 120. In this embodiment, the sleeve 130 is engaged by the gripping element 136 through a push-fit engagement of the handling element 133 of the sleeve 130 within the aperture of the gripping element 136, as described above. The sleeve 130 is therefore configured to unfurl along the length of the catheter 120 as the catheter 120 is withdrawn from the pouch 170. Once the sleeve 130 is completely unfurled it is configured to disengage from the gripping element 136 once a threshold tension is present in the sleeve 130 as described below. In other embodiments any suitable engagement between the gripping element 136 and proximal end 131 of the sleeve 130 may be used.

In this embodiment, the fluid reservoir 140 is an elongate rectangular burstable sachet of water. The fluid reservoir 140 is arranged within the pouch 170 adjacent the right lateral edge 172c. The fluid reservoir 140 has a height equal to between 60-90% the height of the pouch 170, for example 80%, and a width between 5-25% the width of the pouch 170, for example 15%. In other embodiments, many other types of reservoir would be suitable including different shapes and sizes of reservoir, or non-burstable reservoirs and other external sources of fluid. In this embodiment, the wetting fluid is water and interacts with the hydrophilic surface of the catheter 120 to render it lubricious. In other embodiments, other wetting fluids may be used and they may be polar (e.g. water-based) or non-polar (e.g. oil-based) depending on the catheter's surface properties.

Referring to FIG. 7, in this embodiment, the fluid collection bag 110 comprises a front panel 111, a rear panel of identical shape and size (not shown), and a peripheral bond joining the periphery of the panels to form the bag 110. The front panel 111 and rear panel are both transparent or translucent in this embodiment. The peripheral bond defines a base 112, a right lateral edge 113, a left lateral edge 114 and an upper edge 115. The right lateral edge 113 and left lateral edge 114 being defined as the right and left sides of the bag 110 when viewing the bag 110 with the rear panel behind the front panel 111, the base 112 at the bottom of the bag 110 and the upper edge 115 at the top of the bag 110. The peripheral bond thus defines a bag 110 that, when unfolded, is generally rectangular and suitably has a width between the lateral edges of at least 10, 12, 15 or 20 cm. The bag may have a width no more than 25, 20, or 15 cm. Preferably, the width is between 10 and 20 cm, most preferably between 12 and 15 cm. A height from the base 112 to the upper edge 115 may be at least 25, 30, 35 or 40 cm. The height of the bag may be no more than 45, 40, 35 or 30 cm. The height may be between 25 and 40 cm, 30 and 35 cm, or most preferably 33 and 35 cm. The bag may be configured to hold at least 500, 700, or 1000 ml of fluid, and may hold no more than 1000 ml. For example, it may hold at least 700 ml of fluid. The base 112 defines the bottom of the bag 110 in use, and the upper edge 115 the top. However, in other embodiments alternative shapes and sizes could be conceived, such as elliptical or circular panels forming correspondingly shaped bags, or even a tubular bag or bag with more than two panels, e.g. a prism or other polygon.

In this embodiment, the panels comprise a flexible plastics material, for example polypropylene (PP), polyethylene terephthalate (PET), low density polyethylene (LDPE), metalized polyester (MET PET), orientated polypropylene (OPP) or polyvinyl chloride (PVC).

In this embodiment, the inlet 150 is positioned at the corner of the peripheral bond where the upper edge 115 and left lateral edge 114 meet. A neck 151 is formed adjacent to the inlet 150 in the bag 110 by the peripheral bond which comprises two triangular projections 117a, 117b into the bag 110, a left edge projection 117a along the left lateral edge 114 and an upper edge projection 117b along the upper edge 115.

In this embodiment, the left edge projection 117a is a right-angle triangle with its right-angled vertex projecting into the bag 110 and the other vertices along the left lateral edge 114. The left edge projection 117a is approximately one quarter the length of the left lateral edge 114 and begins approximately 10% along the length of the left lateral edge 114 from the upper edge 115. The left edge projection 117a extends from this point approximately 10% across the width of the bag 110 angled slightly below perpendicular to the left lateral edge 114 before turning 90 degrees at the right-angled vertex and returning to meet the left lateral edge 114.

In this embodiment, the left edge projection 117a also comprises a rib 117c spanning from its right-angled vertex to a point on the left lateral edge 114 and approximately midway along the length of the left edge projection 117a. The rib 117c comprises a region which preferentially folds to help with maintaining the shape of the bag 110 both when folded and unfolded and also makes it easier for a user to grasp/grip the bag 110.

In this embodiment, the upper edge projection 117b is a right-angle triangle. The right-angled vertex projecting into the bag 110 and the other vertices along the upper edge 115. The upper edge projection 117b is approximately 90% the length of the upper edge 115 and begins approximately 10% along the length of the upper edge 115 from the left lateral edge 114 and ends at the right lateral edge 113. The upper edge projection 117b extends from where it meets the upper edge 115 approximately 10% down the length of the bag 110 angled slightly right of perpendicular to the upper edge 115 before turning through 90 degrees towards the right lateral edge 113 at the right-angled vertex and then returning to meet the right lateral edge 113.

Thus, as described above, in this embodiment the right-angled vertices of the left edge projection 117a and upper edge projection 117b define the narrowest point of the neck 151.

In this embodiment, the peripheral bond further comprises two finger holes 116 to allow the bag 110 to be easily handled by a user. One finger hold is positioned in the upper edge projection 117b adjacent to its right-angled vertex. The other finger hole 116 is at the corner of the bag 110 where the right lateral edge 113 meets the base 112, and the peripheral bond truncates the corner of the bag 110 to accommodate this finger hole 116. In other embodiments, the finger holes 116 may comprise a series of ridges, or otherwise easily gripped surface or feature to allow the user to easily handle the bag 110. In addition, there may be different numbers of finger holes 116, such as only one or more than two, and they may be placed in different locations on the bag 110.

In this embodiment, the fluid collection bag 110 further comprises a fill level indicator 118 comprising fill markers which allow the user to measure the volume of fluid contained within the fluid collection bag 110. Due to the shape of the bag 110, the cross-sectional area of the bag 110 may vary along the length of the bag 110 from the base 112 to upper edge 115. As such, the fill level indicator may comprise irregularly spaced fill markers to account for this. The fill markers may indicate, either the proportion of the volume of the bag which is filled with fluid, for example 10%, 25%, 50%, 75%, 100% of a safe fill level, or the absolute volume of fluid, for example 100 ml, 200 ml, 300 ml, etc. as required or as is convenient for the user.

Referring to FIGS. 1 and 2, in this embodiment, the packaged catheter assembly 100 is provided with the sheath 119 containing the folded fluid collection bag 110, catheter 120, sleeve 130 and fluid reservoir 140 all contained with the pouch 170 as mentioned above. To wet the catheter 120 so that it is ready for use, the user may release the wetting fluid from the fluid reservoir 140 into the pouch 170. This may be done by any suitable means, but preferably by applying pressure to the outside of the pouch 170 at a region corresponding to the position of the fluid reservoir 140. In embodiments where the pouch is opaque or the fluid reservoir 140 is otherwise not visible, the pouch may comprise a wetting marker to assist the user in locating the fluid reservoir 140.

In this embodiment, the fluid reservoir 140 is configured to rupture under external pressure, creating a tear 141 in the fluid reservoir 140 which release wetting fluid into the pouch 170 where it collects in a wetting region of the pouch 170 to form a pool 1 in the bottom of the pouch 170.

Referring to FIGS. 3 and 4, in this embodiment, the user may then access the catheter 120 by creating an opening in the pouch 170 using the interaction region. The aperture 179 is grasped in one hand, and the pouch 170 below the tear line 178 in another, and the tear-away region 175 is torn from the tear start 176 to the tear stop 177 along the tear line 178. Thus, the pouch 170 is torn between the left lateral edge 172b and right lateral edge 172c at a location below the upper edge 172d, as such, an opening is formed in the pouch 170 that may be used to access its contents. In this embodiment, the tear-away region 175 is fully separated from the pouch 170 to improve access to the contents of the pouch 170. In other embodiments, the tear-away region 175 may not be fully separated from the pouch 170 to reduce the number of separate parts and make the pouch 170 easier to handle. Other embodiments may also feature other ways to access the pouch for example a zip-lock seal.

In this embodiment and referring to FIGS. 3 and 4, the closed catheter assembly (i.e. catheter 120 and fluid collection bag 110) may then be removed from the pouch 170. The sheath 117 and folded fluid collection bag 110 are easily accessible to the user as they are directly accessible via the opening in the pouch 170, the fluid collection bag 110 and catheter 120 may then be withdrawn from the pouch 170 by pulling the sheathed fluid collection bag 110 up and out of the pouch 170.

In this embodiment, prior to withdrawal, only parts of the catheter 120 that are close to the base 172a of the pouch 170 may have been wetted by the pool 1 of wetting fluid. To ensure the parts of the catheter 120 intended to enter the body are adequately wetted, during withdrawal the position of the catheter 120 within the pouch 170 is restricted such that parts of the catheter 120 intended to enter the body must pass through the wetting region and pool 1 of wetting fluid before withdrawal. As described above, in this embodiment this is achieved through a combination of the gripping element 136 restricting the direction of the catheter 120 such that the tangent of the catheter 120 at the gripping element 136 points towards the pool 1 and the resilience of the catheter 120 ensuring that it resists bending over a length equivalent to the distance between the gripping element 136 and pool 1 and thus uncoils as it is pulled out, such that parts of the catheter that are initially above the pool 1 pass through it as the catheter 120 is pulled out of the pouch. As shown in FIGS. 4-6, during withdrawal this leads to the catheter 120 passing through the pool 1, then past the gripping element 136 and finally being withdrawn from the pouch 170. In some embodiments, the aperture of the gripping element 136 may be further configured to ensure excess wetting fluid is removed from the catheter 120 prior to withdrawal.

In this embodiment, during withdrawal of the catheter 120, the sleeve 130 is unfurled, moving from the initial furled configuration of FIGS. 1-3, to an unfurled configuration as shown in FIG. 7. As the fluid collection bag 110 and inlet 150 are moved up and away from the gripping element 136, the distal end 132 of the sleeve 130 also moves upwards. Because the gripping element 136 engages the handling element 133 and proximal end 131 of the sleeve 130, the sleeve 130 therefore unfurls along the catheter 120 as the catheter 120 is removed from the pouch 170. This ensures that the now wetted catheter 120 is protected by the sleeve 130 from dirt from contact with other objects external to the pouch 170 until use. In addition, as the sleeve 130 is located above the gripping element 136 and close to the upper edge 172d of the pouch 170, the outside of the sleeve 130 does not come into contact with the pool of wetting fluid 1. As such, the outside of the sleeve 130 is dry and convenient for the user to handle.

In this embodiment, the absorbent strip 174 advantageously removes moisture from the sheath 119, fluid collection bag 110 and sleeve 130 as it is withdrawn from the pouch 170 which makes these devices more comfortable for the user to handle. In addition, the absorbent strip 174 does not remove moisture from the now wetted catheter 120 as the sleeve 130 creates a barrier that prevents moisture from being absorbed by the strip 174 from the catheter 120.

Referring to FIGS. 4 to 7, in this embodiment, the sheath 119 may then be removed from the folded collection bag 110. Advantageously, the sheath 119 prevents any wetting fluid from contacting the outside of the fluid collection bag 110 or entering between the folds of the folded fluid collection bag 110. This makes the bag 110 easier to open and handle, because if the folds of the bag 110 are wet they can be more likely to stick together or be slippery to touch. The sheath 119 may be removed by sliding it off the folded fluid collection bag 110, or in other embodiments, the sheath 119 may be removed by other means such as tearing or opening the sheath 119 to disengage it from the fluid collection bag 110.

In this embodiment, the fluid collection bag 110 may then be unfolded so that it is ready to accept fluid from the catheter 120. The catheter 120 may be handled via the sleeve 130 and the gripping element 136 disengaged from the sleeve 130. In this embodiment, the gripping element 136 is disengaged by simply pulling the sleeve 130 and catheter 120 away from the pouch 170. In other embodiments, the gripping element 136 may be actively disengaged by twisting the sleeve 130 or otherwise activating the gripping element 136 to release the sleeve 130 for example.

The steps of unsheathing/unfolding the fluid collection bag 110 and disengaging the gripping element 136 as described above may be performed in any order depending on the preference of the user.

Referring to FIG. 7, in this embodiment, the catheter 120 is now ready for use. The sleeve 130 may be used to handle the catheter 120 and the handling element 133 may be used to progressively introduce the proximal end 121 of the catheter 120 into the urethra until fluid flows through the catheter 120 from the bladder. Fluid flowing through the catheter 120 is collected in the fluid collection bag 110 and the volume of fluid contained in the bag can be monitored using the fill level indicator. After use, in this embodiment, as the catheter 120 is withdrawn from the body, the handling element 133 may be used to cover the length of the catheter 120 with the sleeve 130 and the packaged catheter assembly 100 may then be disposed of.

While the embodiments disclosed above is described as a closed catheter assembly in that fluid is collected in a fluid collection bag, other embodiments may not comprise a fluid collection bag and may be open catheter assemblies in that fluid is not collected during use.

The one or more embodiments are described above by way of example only. Many variations are possible without departing from the scope of protection afforded by the appended claims.

1. A packaged catheter assembly comprising a catheter having a proximal end for insertion into the body and a distal end, a pouch comprising a gripping element configured to engage the catheter, and a fluid reservoir configured to release wetting fluid into the pouch so as to wet the catheter, wherein: the catheter and fluid reservoir are arranged within the pouch; the pouch is configured to provide an opening through which the catheter may be withdrawn; the pouch comprises a wetting region in which wetting fluid preferentially collects; the wetting region is distal the opening; and the gripping element is configured to direct the catheter through the wetting region during withdrawal of the catheter from the pouch.

2. The catheter assembly of claim 1 wherein the catheter is a male urinary catheter.

3. The catheter assembly of any preceding claim wherein the catheter is longer than the pouch.

4. The catheter assembly of any preceding claim wherein the catheter is curved between the gripping element and the wetting region.

5. The catheter assembly of any preceding claim wherein the catheter is arranged within the pouch in a coiled configuration.

6. The catheter assembly of any preceding claim wherein the catheter is drawn through the gripping element during withdrawal of the catheter from the pouch.

7. The catheter assembly of any preceding claim wherein the gripping element restricts the direction of an axis of the catheter.

8. The catheter assembly of claim 7 wherein the axis of the catheter is restricted to within 20 degrees of an axis of the gripping element.

9. The catheter assembly of any preceding claim wherein an axis of the gripping element is directed towards the wetting region.

10. The catheter assembly of any preceding claim wherein the gripping element is located outside the wetting region.

11. The catheter assembly of claim 10 wherein the catheter is configured such that during withdrawal of the catheter from the pouch it passes through the wetting region before it reaches the gripping element.

12. The catheter assembly of any preceding claim wherein the catheter is configured to be withdrawn from the pouch in a direction parallel to the axis of the gripping element.

13. The catheter assembly of any preceding claim wherein the wetting region is configured to collect a pool of wetting fluid.

14. The catheter assembly of any preceding claim wherein the catheter is resiliently biased into a straight configuration.

15. The catheter assembly of claim 14 wherein the catheter has a threshold rigidity that ensures it passes through the wetting region.

16. The catheter assembly of claim 15 wherein the threshold rigidity of the catheter is such that the tangent of the catheter resists changing by more than 20 degrees over a length equivalent to the distance between the gripping element and wetting region.

17. The catheter assembly of any preceding claim wherein the gripping element is positioned in an upper half of the pouch and the wetting region is adjacent a base of the pouch.

18. A method of unpackaging a packaged catheter assembly, the packaged catheter assembly comprising a catheter having a proximal end for insertion into the body and a distal end, a pouch comprising a gripping element configured to engage the catheter, and a fluid reservoir configured to release wetting fluid into the pouch so as to wet the catheter, wherein the catheter and fluid reservoir are arranged within the pouch, the method comprising the steps of releasing wetting fluid into the pouch so as to wet the catheter, allowing wetting fluid to collect in a wetting region of the pouch, and withdrawing the catheter from the pouch, wherein the gripping element is configured to direct the catheter through the wetting region during withdrawal of the catheter from the pouch.

19. The method of claim 18 comprising passing the catheter through the wetting region before it reaches the gripping element.

20. The method of any of claims 17 to 19 wherein the catheter assembly is the catheter assembly of any of claims 1 to 16.
